(19) United States
(12) Patent Application Publication
(10) Pub. No.: US 2025/0281302 A1
(43) Pub. Date: Sep. 11, 2025

(54) INTERVERTEBRAL DISC PROSTHESIS AND REVISION METHOD

(71) Applicant: Todd H. Lanman, Beverly Hills, CA (US)

(72) Inventors: Todd H. Lanman, Manhattan Beach, CA (US); Sara Layton, Ladera Ranch, CA (US)

(21) Appl. No.: 19/105,010

(22) PCT Filed: Sep. 23, 2023
(86) PCT No.: PCT/US23/74966

§ 371 Date: Feb. 19, 2025

Related U.S. Application Data
(60) Provisional application No. 63/411,013, filed on Sep. 28, 2022.

(51) Int. Cl. A61F 2/44; A61F 2/30

(52) U.S. Cl. CPC A61F 2/4425; A61F 2002/30227; A61F 2002/443; A61F 2220/0016; A61F 2220/0025; A61F 2250/0004

(57) ABSTRACT

An intervertebral disc prosthesis is described including two endplates and a revisable elastic core. The existing core is removed while leaving the two endplates intact in the spine. A new core is installed between the endplates. The new core can have mechanical properties similar to or different than the originally implanted core. The new design enables the intervertebral disc prosthesis to be revised in situ using minimally invasive surgery.

### CROSS-REFERENCE TO RELATED APPLICATIONS

This claims priority to provisional patent application No. 63/411,013, filed Sep. 28, 2022, and entitled INTERVERTEBRAL DISC PROSTHESIS AND REVISION METHOD, the entirety of which is incorporated by reference in its entirety.

### BACKGROUND OF THE INVENTION

The present invention relates to medical devices and methods. More specifically, the invention relates to intervertebral disc prostheses.

Intervertebral discs are the soft tissue structures located between each of the vertebral bones that make up the vertebral (spinal) column. The discs allow the vertebrae to move relative to one another. The vertebral column and discs are vital anatomical structures, in that they form a central axis that supports the head and torso, allow for movement of the back, and protect the spinal cord, which passes through the vertebrae in proximity to the discs.

Discs often become damaged due to wear and tear or acute injury. For example, discs may bulge (herniate), tear, rupture, degenerate or the like. A bulging disc may press against the spinal cord or a nerve exiting the spinal cord, causing “radicular” pain (pain in one or more extremities caused by impingement of a nerve root).

Degeneration or other damage to a disc may cause a loss of “disc height,” meaning that the natural space between two vertebrae decreases. Decreased disc height may cause a disc to bulge, facet loads to increase, two vertebrae to rub together in an unnatural way and/or increased pressure on certain parts of the vertebrae and/or nerve roots, thus causing pain. In general, chronic and acute damage to intervertebral discs is a common source of back related pain and loss of mobility.

When one or more damaged intervertebral discs cause a patient pain and discomfort, surgery is often required. Traditionally, surgical procedures for treating intervertebral discs have involved discectomy (partial or total removal of a disc), with or without fusion of the two vertebrae adjacent to the disc.

Another surgical approach is disc fusion. Fusion of the two vertebrae is achieved by inserting bone graft material between the two vertebrae such that the two vertebrae and the graft material grow together. Oftentimes, pins, rods, screws, cages and/or the like are inserted between the vertebrae to act as support structures to hold the vertebrae and graft material in place while they permanently fuse together. Although fusion often treats the back pain, it reduces the patient's ability to move, because the back cannot bend or twist at the fused area. In addition, fusion increases stresses at adjacent levels of the spine, potentially accelerating degeneration of these discs.

In an attempt to overcome some of the shortcomings associated with fusion, an alternative approach has been developed, in which an implantable, artificial intervertebral disc (or “disc prosthesis”) is inserted between two vertebrae. Examples of disc prostheses are described in U.S. Pat. No. 9,364,337 to Kim et al., and publication no. 2005/0021146 to de Villiers et al. Although existing disc prostheses provide advantages over traditional treatment methods, improvements are ongoing.

For example, it is not uncommon for the core of an artificial disc to wear or become damaged after implantation in the patient. Although less common, the metal endplates of the artificial disc can also wear or become damaged. For these reasons it is desirable to replace the worn or damaged artificial disc with a new one (i.e., to perform a “revision”).

Revisions, however, can be potentially dangerous (especially in the lumbar spine) for a number of reasons, not the least of which is the blood vessel scarring to the anterior vertebral column. This makes total explantation dangerous, very difficult, and sometimes impossible. Typically, on account of these reasons, the physician must remove the artificial disc and install a fusion, which then restricts patient mobility. Additionally, when replacing a disc from the direct lateral approach, to try and avoid major blood vessels, the physician cannot preserve the bony endplates. Thus, the whole artificial disc, including the endplates and the core, are extracted. Removing a faulty implant in the lumbar region requires extensive bone removal and damages to the bony vertebral body structures so that artificial disc replacement is no longer a possibility. Fusion may be the only available option. As described above, fusions are not desirable.

A need therefore exists for an improved intervertebral disc revision method and prosthesis as described herein that provides for lateral, anterior, oblique, and posterior approaches, and that minimizes damage to the patient.

### SUMMARY OF THE INVENTION

A method of revising an intervertebral disc prosthesis in a spine of a patient comprises: accessing the implanted intervertebral disc prostheses in the spine of the patient; spreading the plates from one another; decoupling the existing core from the plates; removing the existing core from the patient while leaving the plates intact in the spine; placing a new core between the plates; and adjusting the position of the core such that the core is aligned and pivotably engaged with the plates.

The new core may be selected from a plurality of different types of cores, wherein the different types of cores have different types of mechanical properties.

In embodiments, the new core may comprise a different hardness than the initial core.

In embodiments, the surgical approach for replacing the core is the same or different than the initial surgical approach to implant the disc.

In embodiments, the surgical approach to revise the core is a lateral approach, anterior, posterior, or oblique approach using a MIS technique.

In embodiments, the step of spreading is performed with a handheld instrument inserted into a laterally-disposed tool engagement feature arranged on the plates. The handheld instrument can be operable to limit the plates from expanding beyond a threshold distance.

In embodiments, the step of decoupling is performed by inserting a tool through a window in one of the plates, and into an interface formed between the core and the plate such that further advancement of the tool into the interface causes the core to detach from the plate.

In embodiments, the step of decoupling is performed by slidably withdrawing the core along a guide or track in the lower plate, and in some embodiments, withdrawing the core along two parallel channels present in the lower plate.

In embodiments, a connector is unfastened from the edge of the lower plate, freeing the core, prior to slidably withdrawing the core from the lower plate.

In embodiments, the step of placing a new core between the plates is performed by advancing the new core along a guide or track present on the lower plate, and optionally fastening the connector to the lower plate thereby securing the new core in the lower plate.

In another embodiment, an intervertebral disc prosthesis comprises a core and first and second plates locatable about the core.

In embodiments, each plate comprises an outer surface which engages a vertebra, and an inner surface comprising a recess shaped to pivotally receive one of the tabs of the core such that the first and second plates are adapted to articulate with respect to one another and the core when the core is positioned between the first and second plates.

In embodiments, lateral and anterior-disposed tool-engagement features are provided along the periphery for engaging and expanding the endplates from one another using the tool.

In embodiments, the endplates further comprise a peripherally-disposed lip extending vertically from the inner surface, wherein the lip and inner surface form a shallow cavity within which a portion of the core fits.

In embodiments, the endplates further comprise lateral and anterior-disposed windows/cutouts in the lip to provide lateral and anterior access to each interface formed between the core and the inner surface of the endplates after the intervertebral disc prosthesis is implanted in the spine.

In embodiments, each end plate can comprise at least one peg extending vertically from the inner surface, and optionally, the pegs comprise rounded or domed ends.

In embodiments, the core further comprises a vertical channel extending from the upper surface to the lower surface, each vertical channel being arranged along the periphery of the core and aligning with each peg.

In embodiments, the pegs of the upper plate mirror that of the lower plate. In other embodiments, the pegs of the upper plate do not mirror the pegs of the lower plate. The pegs of the upper plate are nonsymmetrical with the pegs of the lower plate.

In embodiments, the pegs and channels are spaced from the lateral and anterior regions of the disc.

In embodiments, the inner surface of each of the endplates is lip-less and the pegs serve to hold the core in place. When the endplates lack a perimeter wall or lip, the core-plate interface is generally accessible along the entire periphery except where a peg is present. In such embodiments, the pegs are preferably located away from the anterior and lateral positions so that the physician may have unobstructed access to the core using either an anterior or lateral approach.

In embodiments, the core has a circular or oval-like profile when viewed from the top.

In embodiments, the core comprises a core hold for receiving and engaging the distal working end of a core tool to separate the core from the plates. In embodiments, the core holds are located along the lateral edge and anterior edge of the core.

In embodiments, at least one of the core and endplates comprises a bioactive agent.

In embodiments, the tool-engagement feature is a hole, slot, key-hole, or combination of hole and slot.

In embodiments, the tab is semi-spherical shaped.

In embodiments, the core is formed of a resilient material comprising a polymer.

In embodiments, at least one of the endplates have an outward bow ranging from 1 to 25 degrees from normal, and in some embodiments, from 10 to 25 degrees from normal. The convex, c-shape or outward bow serves to both (a) secure the endplates in the vertebra as well as (b) accommodate the core tabs for pivot motion.

In some embodiments, the endplates have a slanting outer surface from low to high in the lateral or posterior direction to compensate for defects in patient's spine such as sclerotic or kyphotic defects, respectively.

In some embodiments, an intervertebral disc prosthesis comprises: an upper plate; a lower plate; and a core adapted to be removably installed between the upper plate and the lower plate wherein the core comprises a substantially planar base, a centrally-located superiorly-extending tab, and a peripheral area or tongue-type region circumscribing the tab.

In embodiments, the lower plate comprises a left groove, a right groove, and a cavity between the left groove and the right groove, and wherein the left groove and right groove cooperate with a left portion and right portion of the tongue, respectively, of the base to slidably receive the base of the core in the cavity.

In embodiments, an anterior connector is removably engageable to an anterior side of the lower plate to secure the core in the cavity in the lower plate.

In embodiments, the anterior connector comprises a posterior facing rail, and forms an anteriorly-located groove for receiving an anterior portion of the tongue when the anterior connector is fastened to the lower plate.

In embodiments, the anterior connector further comprises at least one tool engagement feature to receive an end of a tool, and optionally, wherein the tool engagement feature is an elongate slot.

In embodiments, the left groove is formed by a rail of a left-side connector removably engageable to the left side of the lower plate.

In embodiments, the left-side connector further comprises at least one tool engagement feature to receive an end of a tool, and optionally, wherein the tool engagement feature is an elongate slot.

In embodiments, the right groove is formed by a rail of a right-side connector removably engageable to the right side of the lower plate.

In embodiments, the lower plate further comprises a posterior groove adapted to slidably engage a posterior portion of the tongue.

In embodiments, the tab of the core has a semispherical shape and an inner surface of the upper plate comprises a concave recess to pivotably cooperate with the tab when the disc is assembled in the body.

In embodiments, the intervertebral disc prosthesis comprises a profile matching that of a natural disc, or optionally oval.

In embodiments, the upper surface of the upper plate comprises a bow, and optionally at least one fin.

In embodiments, the upper plate is lipless.

In embodiments, at least one of the upper plate and lower plate comprises at least one tool engagement feature on an anterior, a lateral side, or optionally both sides, and wherein the at least one tool engagement feature is operable to receive an end of a tool, and optionally, wherein the tool engagement feature is an elongate slot, hole, or key-hole.

The description, objects and advantages of embodiments of the present invention will become apparent from the detailed description to follow, together with the accompanying drawings.

### BRIEF DESCRIPTION OF THE DRAWINGS

FIG. 1 is a top side perspective view of an intervertebral disc prosthesis in accordance with embodiments of the invention;

FIG. 2 is an enlarged partial side view of the disc prosthesis shown in FIG. 1;

FIG. 3 is a bottom side perspective view of the upper plate shown in FIG. 1;

FIG. 4 is a top side perspective view of the core shown in FIG. 1;

FIGS. 5A-5H sequentially illustrate a procedure for implanting an artificial disc prosthesis between two vertebrae in accordance with an embodiment of the invention;

FIGS. 6A-6H sequentially illustrate a procedure for replacing in situ an existing core of an artificial disc prosthesis with a new core while leaving the upper and lower plates intact in the spine in accordance with an embodiment of the invention;

FIG. 7A is a top side perspective view of an intervertebral disc prosthesis in accordance with another embodiment of the invention;

FIGS. 7B-7H are top, right side, left side, front, rear, bottom, and exploded views, respectively, of the disc shown in FIG. 7A;

FIG. 8A is a top left side perspective view of an intervertebral disc prosthesis in accordance with another embodiment of the invention;

FIG. 8B is a top view of the disc shown in FIG. 8A;

FIG. 9 is a top right side (i.e., patient right side) perspective view of an intervertebral disc prosthesis in accordance with another embodiment of the invention;

FIG. 10 is an exploded view of the intervertebral disc prosthesis shown in FIG. 9;

FIG. 11A is an anterior or front view of the core of the intervertebral disc prosthesis shown in FIG. 9;

FIG. 11B is an anterior or front perspective view of the lower plate of the intervertebral disc prosthesis shown in FIG. 9 without the lateral and anterior connectors;

FIGS. 12-13 are various exploded views of the lower end plate, core, and anterior connector of the intervertebral disc prosthesis shown in FIG. 9;

FIG. 14 is an enlarged anterior view of a portion of the disc assembly shown in FIG. 10; and

FIG. 15 is an enlarged right side view of a portion of disc assembly shown in FIG. 10.

### DETAILED DESCRIPTION OF THE INVENTION

Before the present invention is described in greater detail, it is to be understood that this invention is not limited to particular embodiments described, as such can, of course, vary. It is also to be understood that the terminology used herein is for the purpose of describing particular embodiments only, and is not intended to be limiting, since the scope of the present invention will be limited only by the appended claims. Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges can independently be included in the smaller ranges and are also encompassed within the invention, subject to any specifically excluded limit in the stated range. Where the stated range includes one or both of the limits, ranges excluding either or both of those included limits are also included in the invention. Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described. It is noted that, as used herein and in the appended claims, the singular forms “a”, “an”, and “the” include plural referents unless the context clearly dictates otherwise. It is further noted that, as used herein and in the appended claims, the terms “right-side” and “left-side” can mean the right side and left side, respectively, from the perspective of the patient anatomy unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation. As will be apparent to those of skill in the art upon reading this disclosure, each of the individual embodiments described and illustrated herein has discrete components and features which can be readily separated from or combined with the features of any of the other several embodiments without departing from the scope or spirit of the present invention. Any recited method can be carried out in the order of events recited or in any other order that is logically possible.

All existing subject matter mentioned herein (e.g., publications, patents, patent applications and hardware) is incorporated by reference herein in its entirety except insofar as the subject matter may conflict with that of the present invention (in which case what is present herein shall prevail).

Various embodiments of the present invention generally provide for an intervertebral disc prosthesis, namely, an intervertebral artificial disc, having upper and lower plates disposed about a core.

As described herein, after the artificial disc is implanted in the patient, the existing core may be replaced, in situ, with a new core while leaving the endplates intact. Doing so allows the surgeon to leave most scar tissue in place therefore reducing the recovery time and improving comfort for the patient post-op.

Another advantage of the disc prosthesis and techniques described herein is that the disc prosthesis may be implanted and revised using many different surgical approaches to access the intervertebral disc space including, e.g., anterior, lateral, posterior and posterior lateral approaches. If anterior placement was used for initial implantation, for example, the surgeon may use a lateral minimally invasive surgery (MIS) approach for the revision to avoid reopening the space where scar tissue has formed. Embodiments of the present invention enable a MIS revision because only the core is removed—not the entire implant.

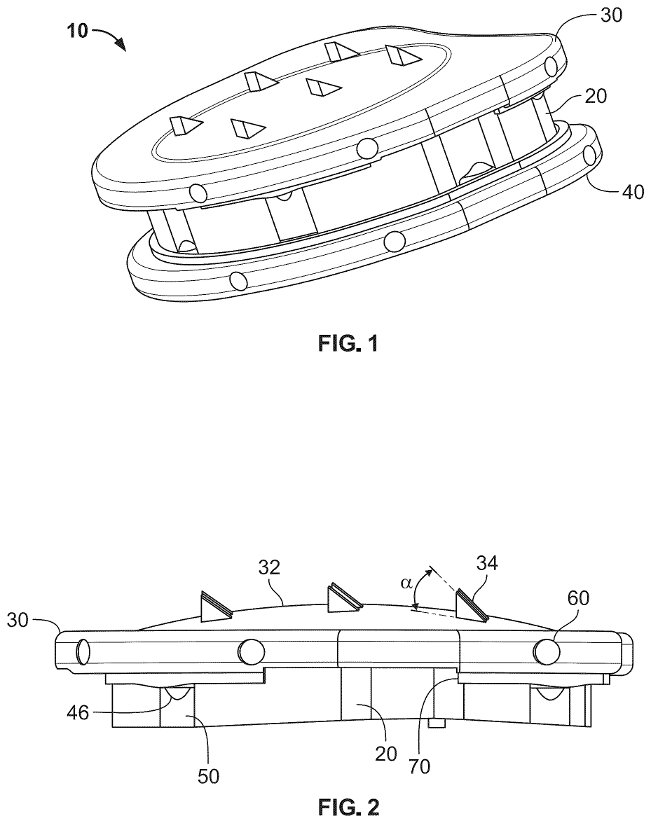

Now, with reference to FIG. 1, a prosthetic disc 10 is shown comprising a core 20 arranged between an upper plate 30 and lower plate 40 according to embodiments of the present invention.

### Endplates

The upper plate 30 may be constructed from any suitable metal, alloy or combination of metals or alloys, such as but not limited to cobalt chrome molybdenum, titanium (such as grade 5 titanium), stainless steel and/or the like. Additionally, the surfaces of the plate may be treated to make the plate adhere to the bone body or core as the case may be. For example, the outer surface can be treated by aluminum oxide blasting and optionally followed by a titanium plasma spray. Indeed, a wide range of suitable metals or combinations of metals and surface treatments may be used for improving strength, visibility under fluoroscopy or MRI, biocompatibility, etc. In embodiments, the endplates are treated or coated with a bioactive agent such as an antibiotic agent to limit, prevent, or treat infection. All combinations of materials are contemplated within the scope of the present invention.

### (a) Endplate Outer Surface

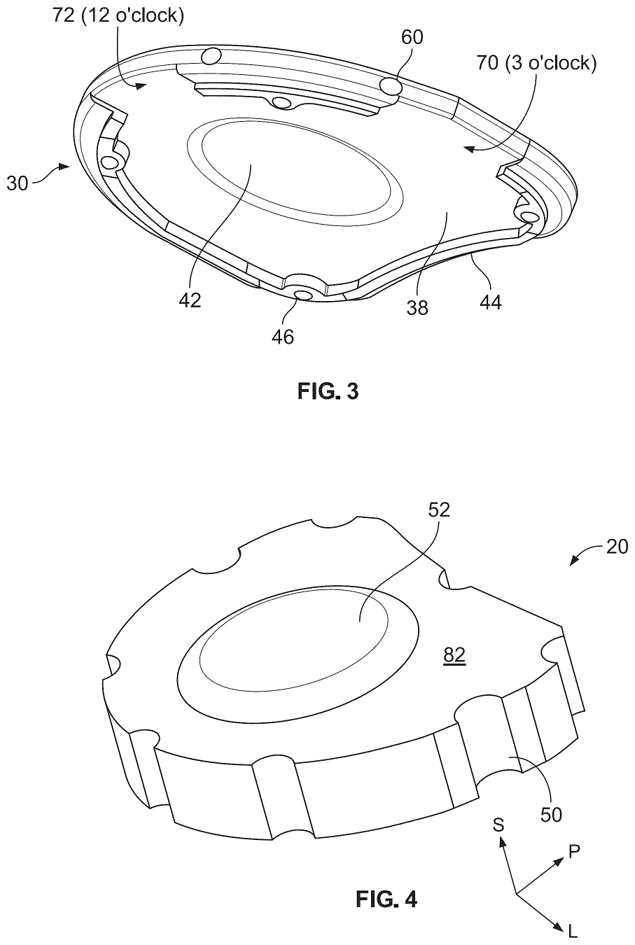

With reference to FIG. 2, the outer surface 32 of the upper plate 30 is shown having a slight domed shape, serving to fit with the anatomy of the vertebrae and secure implantation.

The outer surface 32 of the end plate 30 may also carry one or more vertical fins 34, extending in an anterior-posterior direction. In the embodiment shown in FIGS. 1-2, two parallel rows of fins are shown. Each row is shown having three discrete fins. Each fin decreases in height from the anterior to posterior at a slope angle alpha (α). The fin orientation serves to decrease the amount of bone and tissue required to be scraped away for implantation. These anterior-posterior backwards-facing fins 34 allow for easy insertion into the desired region while minimizing the “caught” tissue during insertion. This design is anticipated to minimize the toll on patients' post-surgery while still obtaining a secure attachment. In embodiments, angle (α) is less than 90, more preferably less than 70, and in preferred embodiments ranges from 35-65 degrees.

The fins 34 may be integrally formed with or otherwise secured to the outer surface of the end plate. Additionally, in some embodiments, such as smaller discs for cervical insertion, the fins may be omitted altogether.

End plate 30 may also include one or more surface features and/or materials to enhance attachment of the disc prosthesis 10 to vertebral bone (not shown). For example, the outer surface may be machined to have grooves, dimples, texture, serrations or other surface features for promoting adhesion of the upper plate to a vertebra. Additionally, the outer surface may be provided with a rough micro-finish formed by blasting. In some embodiments, the outer surface may also be plasma sprayed to further enhance attachment of the outer surface 18 to vertebral bone.

Although the end plate is preferably of one-piece construction, when the end plate includes an assembly of multiple components, any suitable technique may be used to couple materials together, such as snap fitting, slip fitting, lamination, interference fitting, use of adhesives, welding and/or the like. Any other suitable combination of materials and coatings may be employed in various embodiments of the invention.

### (b) Endplate Inner Surface

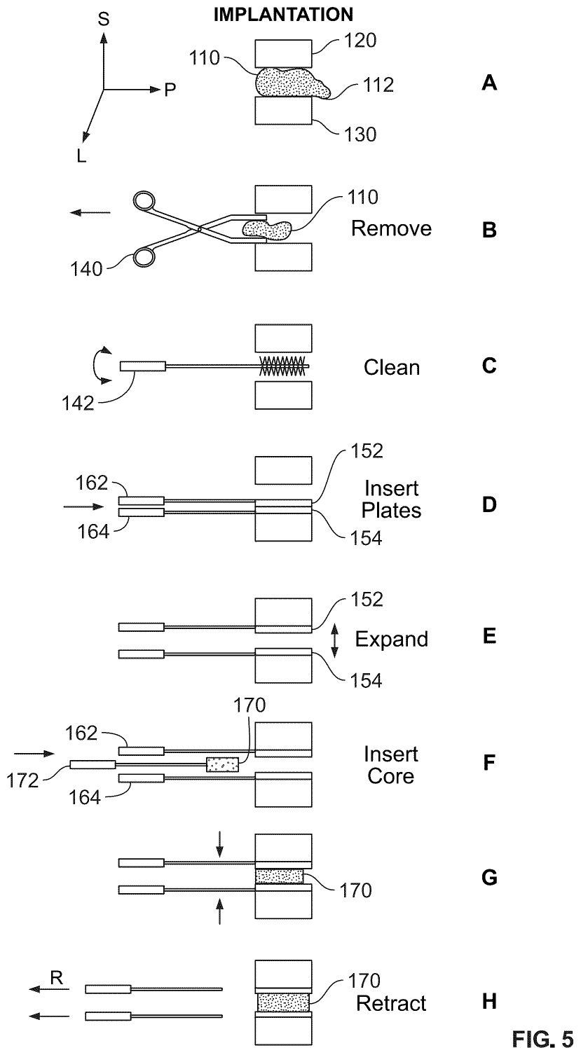

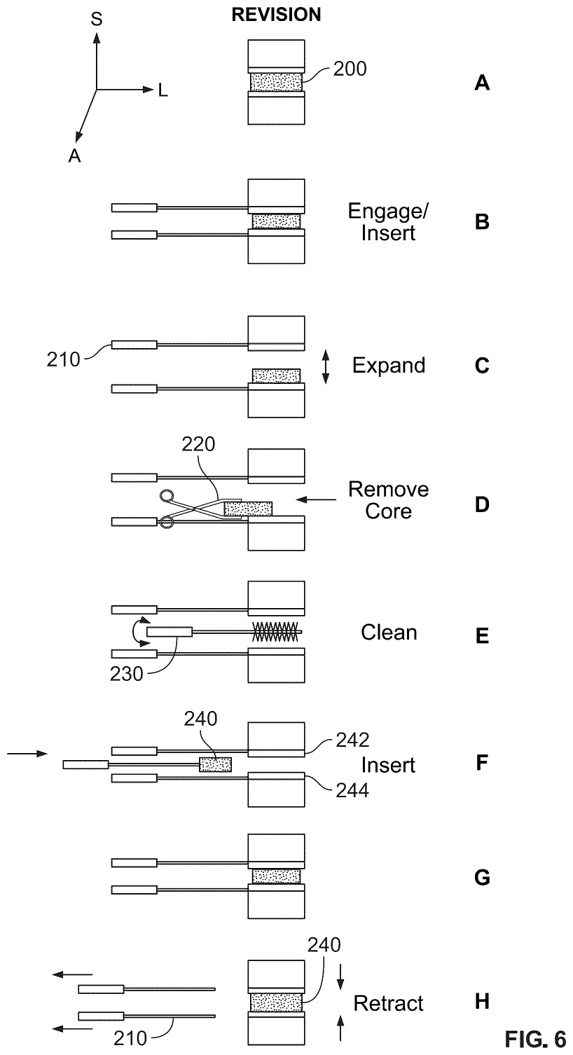

FIG. 3 is a bottom side perspective view of the upper endplate 30 in which inner surface 38 is visible. A concave trough or recess 42 is centrally located that is adapted to register (and rotatably cooperate) with a convex tab 52 protruding from the top of the core 20, discussed further herein in connection with FIG. 4.

FIG. 3 also shows lip 44 and pegs 46 arranged about the periphery of the inner surface 38. Lip vertically extends a height (H) from the inner surface and forms a perimeter wall or boundary sized to receive an upper portion of the core. In embodiments, the height of the lip ranges from 0.5 to 1 mm, and in a preferred embodiment, the height ranges from 0.6 to 0.8 mm.

Pegs 46 are shown extending from the inner surface and spaced apart along the periphery of the inner surface 38. The pegs 46 align with vertical channels 50 formed in the core 20, and prevent free relative movement between the components during torsion and twisting. The pegs preferably have slightly domed ends and act as a stopper in conjunction with the opposing endplate. In the case of hyperextension and movement, the peg will contact an opposing plate (or peg if present) and prevent further pivotal movement.

Although the disc 10 shown in FIGS. 1-4 includes, collectively, 8 pegs and 8 channels, the invention is not intended to be so limited. The number of pegs and channels may vary.

The superior endplate 30 shown in FIG. 3 also includes several tool engagement features (e.g., holes) 60 for receiving an end of a tool, and to spread the discs apart for insertion or revision of the core, described herein. The tool engagement features 60 are spaced along the peripheral edge of the endplate for both lateral or anterior access. Particularly, the tool engagement features are provided at both anterior positions and lateral positions so the endplate may be manipulated from either a lateral or anterior approach.

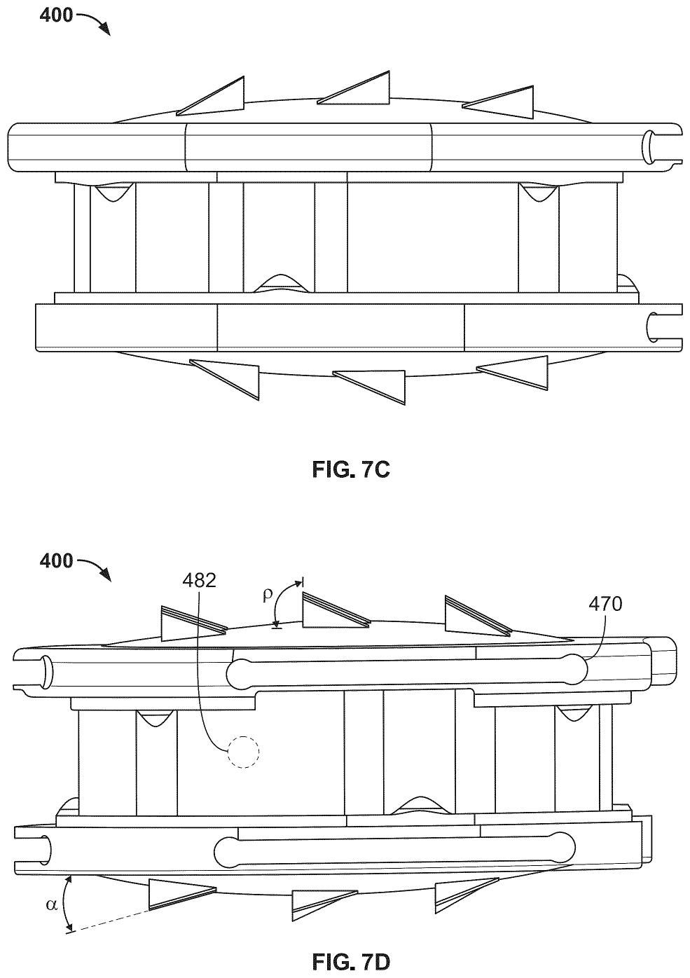

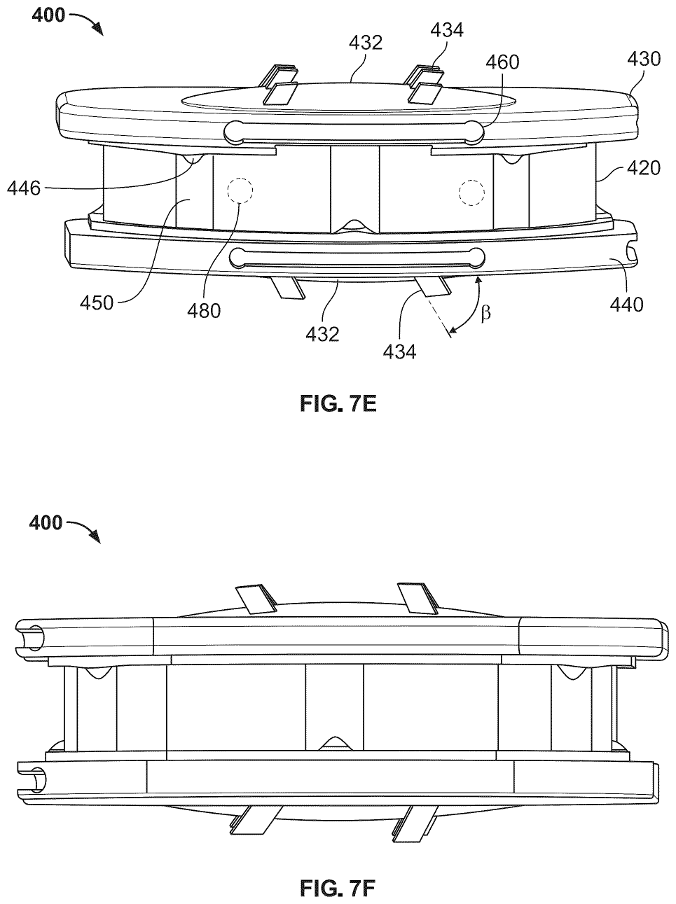

The tool engaging feature may vary in design. In embodiments, the tool engaging feature is a hole, socket, or slot for receiving a distal end of an instrument as described further herein with reference to FIGS. 7A-7H.

The superior endplate 30 shown in FIG. 3 also includes several windows or cutouts 70,72 for providing access to the core/endplate interface, discussed further herein. The cutouts 70,72 are preferably disposed at anterior and lateral (e.g., 12, 3, and 9 o'clock) positions so that the core may be detached from the endplate from either a lateral, anterior, oblique, or posterior approach. The windows allow for the surgeon to slide a tool therethrough, access the interface/plane between the core and the endplate, and detach or release the core 20 from the plate.

The shape of the window 70 may vary. In embodiments, the window is rectangular or square. However, in alternative embodiments, the window is curved or semicircular. Additionally, a set of lateral windows can be provided along the lip of the endplate. For example, if a vertical peg is present at the 3 o'clock position, two lateral windows may be provided-one on each side of the peg corresponding to, e.g., the 2 and 4 o'clock positions. It is to be understood that the features described herein (e.g., pegs, windows, channels, tabs, tool engagement features, fins) may be positioned or arranged and combined with one another in any logical manner except where doing so is exclusive to one another.

### (c) Opposing Endplate

In embodiments, the lower (or inferior) plate 40 is designed similar to the upper plate 30. The lower plate can include a domed outer surface, fins, an inner surface defining a concave recess, a peripheral wall, peripheral pegs, anterior and lateral windows, and a plurality of tool engagement features arranged along the edge of the plate as described above.

In embodiments, the arrangement of the pegs on each of the opposing endplates is identical to (or mirrors) the other. In other embodiments, the arrangement of the pegs on the upper plate is different than that of the lower plate. For example, in one embodiment, lower plate 40 includes pegs at about the 12, 3, 6 and 9 o'clock positions and the upper plate 30 includes pegs at about the 1:30, 4:30, 7:30, and 10:30 o'clock positions where the 12 and 3 o'clock positions refer generally to the anterior 72 and left side lateral 70 cutout locations, respectively, as shown in FIG. 3. In embodiments, only the upper plate includes cutouts or windows. However, in other embodiments, only the lower plate includes cutouts and, in some embodiments, both the upper and lower plate include cutouts.

In embodiments, the endplates are identical to one another. For example, in one embodiment, both the upper and lower endplates have a lateral window or opening for detaching the core from the endplates. The pegs on each of the opposing endplates may also mirror each other.

Additionally, in embodiments, the pegs are arranged such that the cutouts on both the upper and lower endplates remain unblocked. For example, the pegs on both the upper and lower plates are arranged to be slightly off-set from the windows such that a tool may be inserted through the window and into the interface between the core and the endplate without contacting or otherwise being obstructed by a peg. For example, in one embodiment, lower plate 40 includes pegs at about the 2, 5, 8 and 11 o'clock positions and the upper plate 30 includes pegs at about the 1, 4, 7, and 10 o'clock positions, and cutouts are provided at the 12, 3, and optionally 6, and 9 o'clock positions. Such an arrangement limits pivoting motion as described above, and provides unobstructed tool access to the core/plate interface on both inferior side and superior side of the implant. Indeed, the arrangement of the pegs and windows may vary widely and is not intended to be limited except where recited in any appended claims.

### Core

FIG. 4 shows an upper side front perspective view of a core 20 in accordance with embodiments of the invention. The core 20 is shown having an upper surface 82 and semi-spherical tab 52 protruding therefrom. The semi-spherical tab 52 is adapted to register with the concave recess 42 described above, permitting some degree of torsion, rotation and pivot to the disc assembly. Once implanted, the complementary and cooperating spherical surfaces of the plates and core allow the plates to slide or articulate over the selected core through a fairly large range of angles and in all directions or degrees of freedom, including rotation.

The bottom or lower surface (not shown) of the core 20 may be identical to the upper surface 82. The lower surface thus can have a semi-spherical tab that torsionally cooperates with the recess in the lower end plate similar to the mechanical cooperation between tab 52 and recess 42. The lower surface may have any one or more of the upper surface features described herein. However, in some embodiments, the lower surface of the core is flat or planar and lacks the tab, in which case only the upper plate is operable to substantially pivot.

The core is shown having an oval or circular-like profile when viewed from the top. The degree of eccentricity may be characterized by an aspect of the minor axis to the major axis. In embodiments, the aspect ratio ranges from 0.5 to 0.8, and more preferably from 0.6 to 0.7.

When the endplates include a lip or perimeter wall, the core has an outer periphery or diameter that is sized to fit within the interior cavity or chamber defined by the wall/lip 44 of the upper and lower endplates. Optionally, in embodiments, the diameter of the core is very slightly larger than the inner diameter of the cavity formed by the wall of the endplates so that an interference fit is formed.

The core is also shown including channels or grooves 50 to accommodate the pegs 46 extending from the endplates. In the embodiment shown in FIGS. 1-4, there are an equal number of channels as pegs. That is, there is one peg (whether extending from the upper or lower plate) per channel. However, in embodiments, the number of pegs and channels is unequal. For example, there may be more channels present in the core than pegs. Some channels may not accommodate a peg. Additionally, and although not shown, the channels and pegs may be offset from the edge instead of along the edge as shown in FIGS. 1-4. For example, through-holes can be added to extend through the core to receive the pegs in lieu of the grooves 50 shown in FIGS. 1-4.

Preferably, however, the disc comprises multiple pegs and channels about the circumference of the disc. The pegs 46 serve a number of functions including maintaining the position of the core, and limiting pivotal motion between the plates and the core. Additionally, in embodiments, the pegs are smooth with a gently curved (e.g., domed shape) head to reduce friction between the pegs and the channels. According to embodiments of the present invention, it is desirable for the plates to easily detach from the core, without sticking or excess friction.

In embodiments, one or more core holds are arranged along the core edge. Examples of core holds include holes, slots (horizontal, vertical or angular) or combinations of the same. With reference to FIGS. 7D, 7E, for example, a lateral core hold 482 and an anterior core hold 480 are shown. The core holds can be used to detach the core from the endplates during a revision procedure in addition to or, in lieu of, inserting a spacing tool through the endplate cutouts 70,72. In embodiments, the core holds and endplate tool engagement features allow the physician to separate the core from the endplate without endplate cutouts 70, 72.

Dimensions of the core may vary. In embodiments, the height of the core (excluding the tab) ranges from 8 mm to 14 mm. In embodiments, the height of the tab ranges from 0.2 to 0.8 mm. In embodiments, the diameter of tab ranges from 10 to 20 mm.

The core 20 may be formed of a wide range of materials. Exemplary categories of types of materials include elastic polymers. Exemplary materials for the core are thermoplastic polymers such as polycarbonate urethane (PCU). Additionally, in some embodiments, a coating or sheath may be applied over the core. For example, the core may comprise combinations of materials to achieve a hardness, biocompatibility, and adhesive or hydrophilic property. In embodiments, the core is formed, embedded, coated, or otherwise treated with a bioactive agent such as an antibiotic agent to limit, prevent, or treat infection.

As discussed herein, in embodiments, a new core is installed in place of the old core. The new core may be selected from different types of cores based on the patient's diagnosis or needs. In embodiments, the new core is selected based on one or more mechanical properties. Examples of mechanical properties include, without limitation, elasticity, hardness, height or thickness. For example, the clastic modulus may range from 10 MPa to 50 MPa. The hardness may range from 75A-75D according to the shore hardness scale. The height may range from 5-15 mm, and in some embodiments, 8-12 mm. In embodiments, and as discussed herein, a disc revision method may replace an existing artificial (perhaps broken or worn out) core with a new core. The new core may have the same or different properties than the original properties of the existing artificial core. For example, the new core may have a different hardness, modulus, or height than the original properties of the existing artificial core.

In embodiments of the invention, a kit includes a plurality of cores of a varying hardness. For example, a kit may include a first set of cores of a first hardness and a second set of cores of a second hardness different from the first hardness. The first hardness may vary from the second hardness 5-30 percent, and in some embodiments, 10-15 percent. The kit may also include one or more inferior plates, one or more superior endplates, and instructions for use (IFU) documentation for implanting and revising the implant and cores as described herein.

### Procedure Implantation

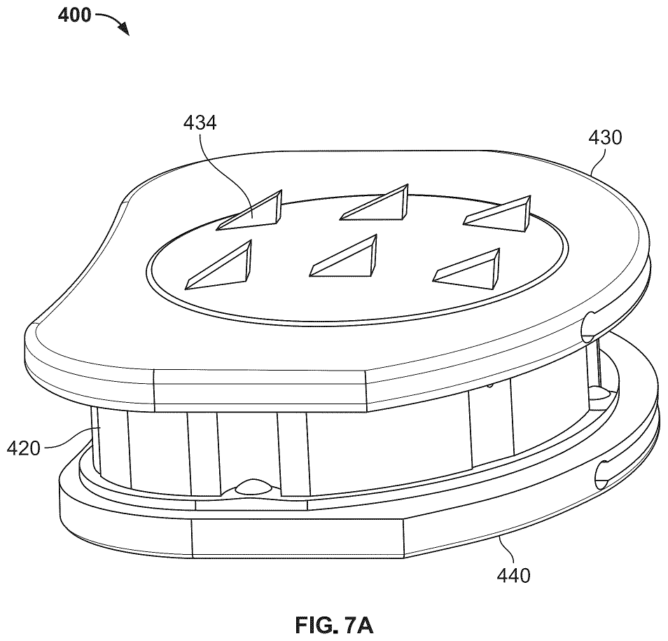

FIGS. 5A-5H sequentially illustrate a procedure for implanting an intervertebral disc prosthesis using an anterior-posterior approach in accordance with an embodiment of the invention.

With reference to FIG. 5A, a native intervertebral disc 110 with a bulge 112 is shown between opposing vertebrae 120,130.

To obtain access to the intervertebral space, an incision is initially carried out along the midline of the abdominal. Dissection is carried through the fatty tissue to the spinal abdominal muscles which run vertically, therefore the incision is carried out between the two abdominal flexor muscles. The cut is then continued through the fascia. Underneath the fascia is the peritoneal cavity where the intestines are within the peritoneal sac. This sac is not opened and the dissection is done around the intestines which are moved out of the way to expose the front of the spine. Directly in front of the spine are blood vessels: the aorta and common iliac arteries and the vena cava and the two iliac veins. The vascular surgeon mobilizes and moves the arteries and veins out of the way of the spine to provide direct access to the spine.

With reference to FIG. 5B, once the spine surgeon has direct access to the spine and intervertebral space, the disc is removed using a forceps 140 or another tool.

With reference to FIG. 5C, the surfaces of the bone are cleared of any cartilage with a brush 142 or another tool.

Next, the upper and lower endplates 152, 154 of the disc are inserted into the cleared space between the vertebral 120, 130. This is illustrated in FIG. 5D in which two probes 162, 164 are shown advancing the upper and lower endplates 152, 154 respectively into the cleared space. The probes may comprise a distal end adapted to engage the tool engagement features described above in connection with FIGS. 1-4 of the disc 10.

Although the plates 152, 154 are shown being manipulated by two discrete probe tools 162, 164, in embodiments, a single instrument is adapted to hold both plates. In a preferred embodiment, a single instrument includes pivotable jaws adapted to expand or spread the plates apart. Optionally, the jaws are spring-loaded and biased to spread apart. Marks or other indicia can be present on the instrument which correlate to the degree of expansion of the jaws. Additionally, an adjustable mechanical stop can be set to limit the distance that the plates can be spread apart.

With reference to FIG. 5E, the two probes 162, 164 are shown inserted into the anterior expansion holes on both the inferior and superior endplates 152, 154. The set of probes can apply forces (e.g., equal and opposite forces) to create an opening between the endplates.

FIG. 5F shows the core 170 being anteriorly advanced by tool 172 into the enlarged space between the opposing endplates 152, 154. The tool 172 may be a probe or forceps or another type of tool which can engage and slide the core into position. In embodiments, the tool 172 has a distal edge that can engage a core hold located along the anterior edge (e.g., feature 480, FIG. 7E).

FIG. 5G illustrates the core 170 in place between the endplates with the core-delivery tool 172 removed. Optionally, the probes may be manipulated towards one another to lock the core in place.

FIG. 5H shows the two probes being retracted (R), allowing the endplates to close over the core and thus, locking the disc into place.

Preferably, imaging is performed to ensure optimal placement of the artificial disc. All layers are then closed sequentially upon exiting the spine.

During and/or after insertion, the vertebrae, facets, adjacent ligaments and soft tissues are allowed to move together to hold the disc in place. The features on the outer surfaces of the plates locate against the opposing vertebrae. The fins provide initial stability and fixation for the disc. With passage of time, enhanced by the presence of any surface coating, firm connection between the plates and the vertebrae will be achieved as bone tissue grows over the fins and outer surfaces.

### Procedure Core Revision

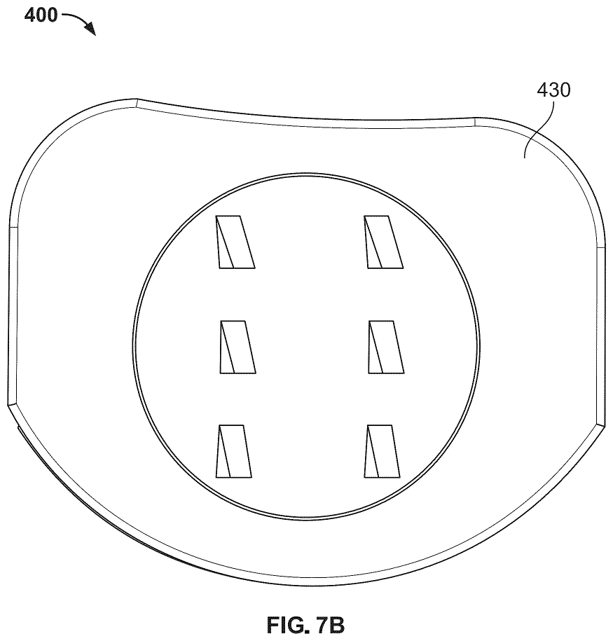

FIGS. 6A-6H sequentially illustrate a procedure for revising an intervertebral artificial disc using a lateral (preferably a MIS lateral) approach in accordance with an embodiment of the invention.

With reference to FIG. 6A, the surgeon obtains access via MIS to the spine as is known to those of skill in the art.

With reference to FIG. 6B, two probes are shown inserted into the cut space and engaging the two endplates of the artificial disc, particularly, the ends of the probes 210 are engaging the lateral expansion tool engagement features (e.g., holes) described above in connection with FIGS. 1-4.

With reference to FIG. 6C, forces (e.g., equal yet opposite forces) are applied to unlock and spread the endplates apart. This step may be performed similar to that described above in connection with FIGS. 5A-5H.

With reference to FIG. 6D, once the endplates are spread apart, a gripping tool 220 can be inserted into the patient to detach, grab, and remove the existing artificial core 200 while preserving the endplates.

The structure of the gripping tool may vary. In embodiments, the gripping tool 220 has opposing jaws to clamp onto the core 200. Finger grips (e.g., rings or loops) are provided to actuate the jaws. In a preferred embodiment, the distal clamping jaw members are planar or spatula-shaped so as to be capable of sliding into the interface between the core 200 and the plates 242, 244. The planar or spatula shaped jaws are gently advanced to separate or space the core from the endplates. Then, the jaws are actuated to clamp or grip the core.

Optionally, other tools may be provided to separate the core from the plates. For example, a spacing tool can include a handle, shaft, and distal end comprising a thin planar body or dull blade. The spacing tool can be inserted into the interface to separate the core from either endplate if necessary. As described herein, in embodiments, the spacing tool can be advanced through a cutout in either the bottom or top endplate to reach the interface.

Without intending to be bound by theory, the previously installed artificial (e.g., used, worn, or damaged) core can adhere to either or both of the endplates 242, 244. In embodiments, the method further includes the step of observing to which plate the core remains affixed. The physician then actively separates the core from either the upper or lower plate. In preferred embodiments, each plate includes unobstructed access (e.g., a window) to the interface for the physician to insert their spacing or gripping tool. In preferred embodiments, the pegs/channels are arranged at locations along the circumference of the core so as not to interfere with the tool path through the window. Non-limiting exemplary locations for the channels/pegs include the 1, 2, 4, 5, 7, 8, 10, 11 o'clock positions. The pegs are preferably evenly distributed about the perimeter of each plate such that there is even spacing between adjacent pegs and only one peg per channel. As described herein, the pegs can serve not only to locate the core relative to the plates, but also to limit the degree that the endplates pivot during use.

In another embodiment, a core tool comprises a distal probe. The probe can be advanced into tool engagement features present in the core (e.g., core holds 480, 482 of FIGS. 7D, 7E) to engage the core. Examples of core holds include slots, holes, or combinations of the same along the edge of the core. The physician can manipulate the handle of the core tool to separate the core from the endplates. The core is wrested from the endplates. In embodiments, combinations of tools can be used to separate the core from the plates.

It is also to be understood that in some embodiments, and although uncommon, the core may not adhere to a plate when the plates are spread. In such instances, the physician can grab the ‘free’ core with a gripping tool such as a forceps.

After the core is spaced from the plates, the physician grips the core and retracts it from the patient while the endplates are left intact in the spine.

With reference to FIG. 6E, after the core is removed, a cleaning device 230 is shown being inserted to remove any debris and to prepare the endplates to ensure surface-core readiness.

FIGS. 6F-6G show new core 240 being laterally advanced between the existing endplates 242, 244.

With reference to FIG. 6H, the force on the expansion probes 210 is retracted, and the new core 240 is locked into place.

If desired, the core and plates may be adjusted to engage the mating components (e.g., pegs with channels, tab with recess, etc.). The tools described above may be employed for this adjusting step.

Imaging is preferably performed to ensure optimal placement. All layers are then closed sequentially upon exiting the spine.

Although the procedures described in FIGS. 5-6 include an anterior approach followed by a lateral approach, the approaches may vary for the implantation and revision. For example, the physician may perform an initial whole disc implantation from the side (lateral) and perform the core replacement from the anterior.

Additionally, one type of approach may be followed by the same type of approach for the revision. For example, for cervical disc replacement, the physician may opt to revise the artificial core using an anterior approach regardless of whether the initial implantation was performed using an anterior approach. However, for lumbar disc replacement, the buildup of scar tissue following the initial implantation makes taking the same approach more difficult and the physician can opt to revise the core using a different approach.

### Alternative Embodiments

In alternative embodiments of the invention, the arrangement of the fins on the disc may vary. For example, the angle that the fin makes with the endplate (namely, tilt angle) may vary. With reference to FIGS. 7A-7E, and with particular reference to the view shown in FIG. 7E, an intervertebral disc prosthesis 400 is shown with fins 434 extending from the domed surface 432 of the end plates 430, 440 at an angle (B) less than 90 degrees to facilitate a core 420 revision procedure using a lateral approach as described above with reference to FIGS. 6A-6H. All fins 434 are shown tilted in the same lateral direction. An exemplary range for the tilt angle (B) is 40-70 degrees, and in embodiments, about 50-65 degrees, and in a preferred embodiment about 55-60 degrees. Without intending to be bound to theory, arranging all the fins with an acute lateral tilt angle serves to (1) facilitate a smooth less traumatic installation of the endplates using a lateral approach, and (2) maintain the endplates in place during a core replacement using a lateral approach. The titled face of the fins tends to grip the tissue/bone as the core is retracted laterally.

Intervertebral disc prosthesis 400 is also shown having elongate tool slots 460 for receiving a tool to manipulate the endplates relative to core 420. Not shown, endplate tool may comprise a planar or spatula-like tip adapted to couple with slots 460. The elongate slot 460 provides a larger target area for the instrument to engage than a hole. Although a slot is shown in FIGS. 7A-7H, it is to be understood that a wide range of tools and tool engagement features may be incorporated into the invention except where excluded from any appended claims.

With reference to the top and left side views of the disc prosthesis 400 shown in FIGS. 7B,7D, respectively, it can be seen that tool slots 470 are arranged on the same side that the fins are tilted. In the embodiment shown in FIG. 7B, the fins are tilted to the patient left, and the tool slot is on the patient left side of the disc prosthesis 400. However, it is to be understood that in other embodiments, the fins may be tilted to the patient right.

Optionally, and with reference to FIG. 7D, the anteriorly-disposed face of the fins may form an angle (ρ) with the surface of the endplate less than or equal to 90 degrees, and in embodiments, angle (ρ) ranges from 70-85 degrees. Fin slope angle (α) can also vary and preferably ranges as stated above in connection with FIG. 2.

As mentioned herein, arranging all the backward-facing fins with a tilt angle towards the lateral direction serves to facilitate installation of the endplates using a lateral approach, benefiting the patient by reducing trauma and injury to the tissue. What is more, in the event of revising or replacing the core as described herein, providing the fins with a tilt in the lateral direction serves to maintain the endplates in place while retracting the core. Furthermore, elongate tool slots or holes are arranged on the same side as the fin tilt to allow for manipulation of the plates from the lateral approach. Additionally, in embodiments, tool slots and tool holes are not present on the rear and side opposite the fin tilt.

Additionally, the orientation and number of rows of fins may vary widely. For example, the rows of fins may be rotated away from the anterior-posterior axis to align along another axis such as, e.g., the lateral-lateral axis.

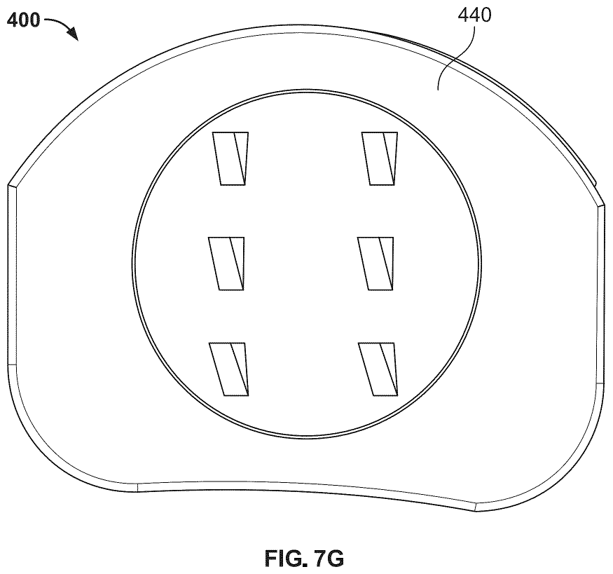

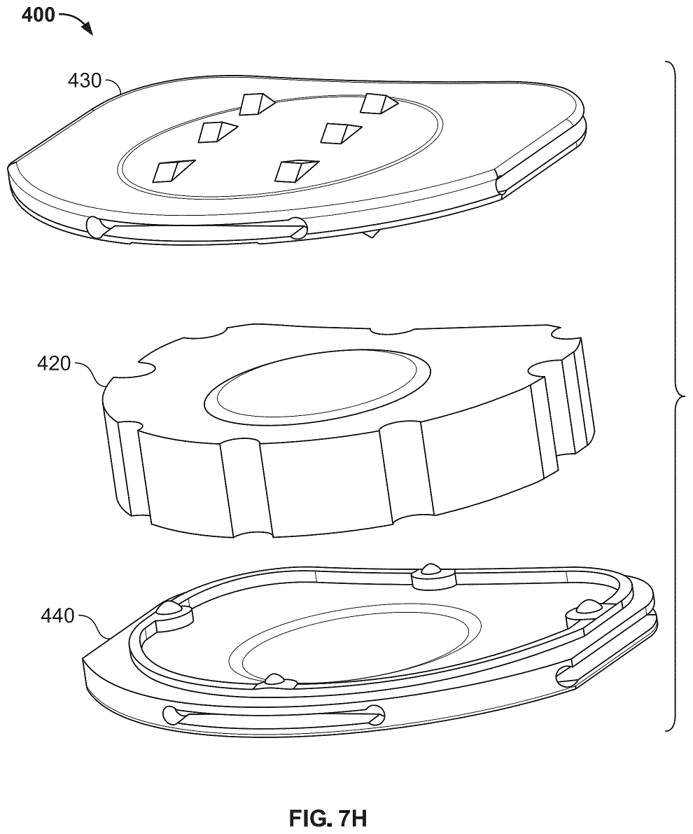

Additionally, the number of fins per row may vary. For example, with reference to FIGS. 8A-8B, a disc prosthesis 500 is shown including four fins. The fins are tilted to the left side for facilitating a disc replacement surgery using a lateral approach. In preferred embodiments, the disc prosthesis includes 1 to 10 fins, more preferably, 4-8 fins, and in some embodiments, 6 fins arranged in a 2 by 3 array. In some embodiments, the fins are equidistant from one another. In some embodiments, the lateral rows of fins are equidistant from one another. In some embodiments, rows of fins formed along the lateral-lateral axis are spaced a greater distance from one another than the columns of fins along the anterior-posterior axis. Additionally, in preferred embodiments, the array of fins is located on the domed surface, and not the flat surface of the endplates.

Additionally, in embodiments, and although not shown, the upper surface 82 of the core 20 can include a core detachment feature such as a laterally-disposed groove that is adapted to receive a tool or instrument (e.g., a core/plate separation probe) using a lateral approach. Preferably, the core detachment feature extends radially along the upper surface 82 from the outer edge towards the center tab 52 such that it can be accessed from the disc edge. If a lip 44 is present on the endplate 30, the entrance to the detachment feature or target can have a depth dimension greater than the height of the endplate lip so that at least a portion of the entrance to the groove remains unblocked by the lip and is accessible to the physician from the disc edge.

Preferably, the entrance to the core detachment feature is arranged at lateral and anterior locations of the disc 10. However, the core detachment feature may be otherwise arranged and extend in other directions including, without limitation, posterior-anterior, oblique, etc. Additionally, if a window 70, 72 is present in the endplate 30 as described above, the groove entrance may be located or aligned with the window. Optionally, the groove decreases in depth from the core edge to the center. The more the physician advances the tool into the core detachment feature, the more pressure is exerted on the core and plate to separate. The core detachment feature is intended to facilitate locating and penetrating the interface between the core 20 and endplate 30 in order to separate the core from the endplate as described herein. Such a feature provides a larger target for the physician to penetrate.

### Core Guide and Locking Connectors

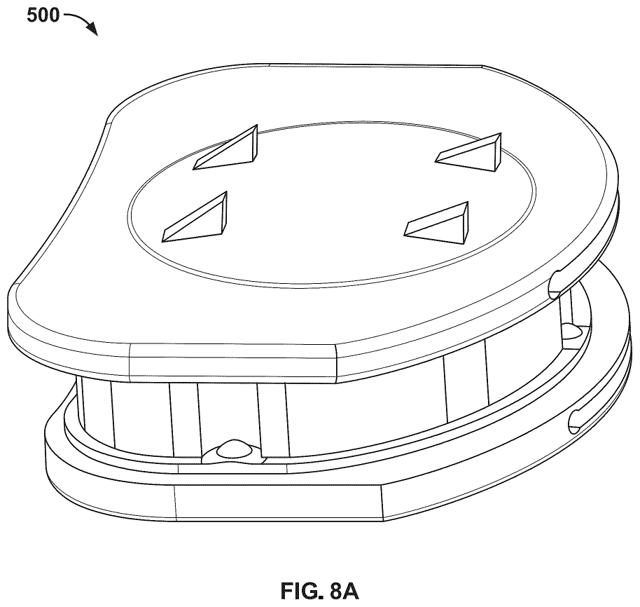

With reference to FIG. 9, an intervertebral prosthetic disc 600 in accordance with another embodiment of the invention is shown. Similar to the embodiments described above, intervertebral prosthetic disc 600 generally comprises a revisable core 620 arranged between an upper plate 630 and lower plate 640. The upper plate 630 (and optionally lower plate 640) has a dome-shaped outer surface 632 and a plurality of fins 634 serving to engage the vertebral bone.

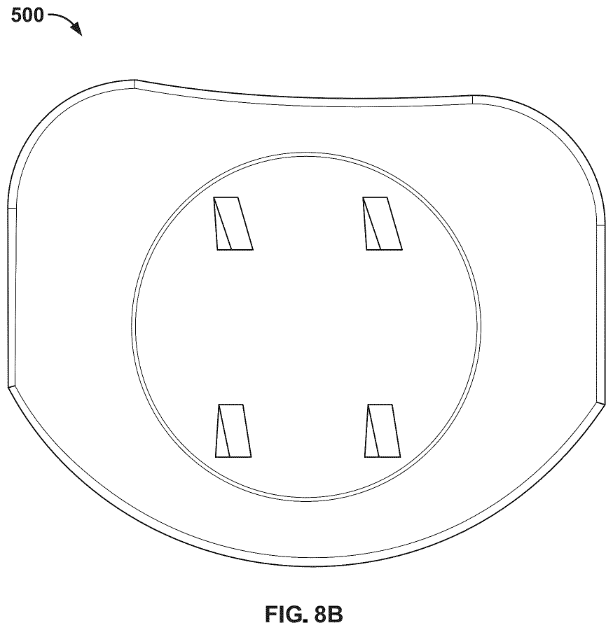

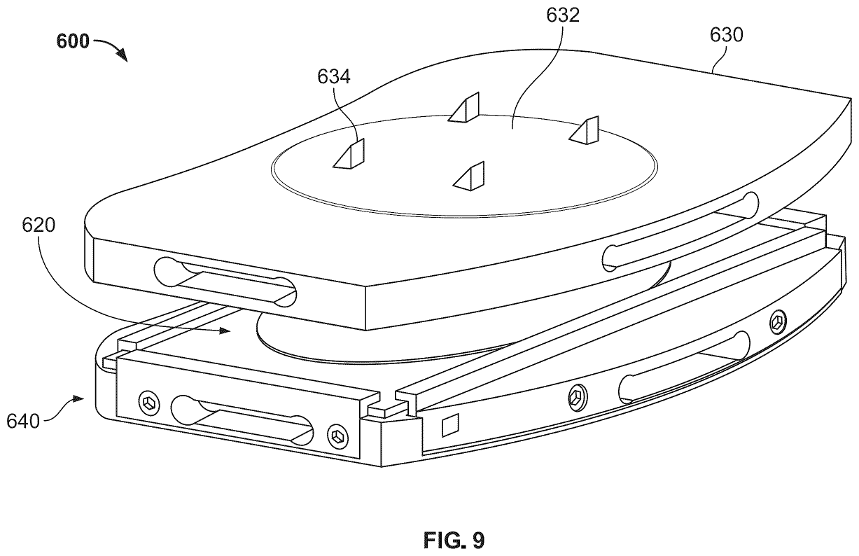

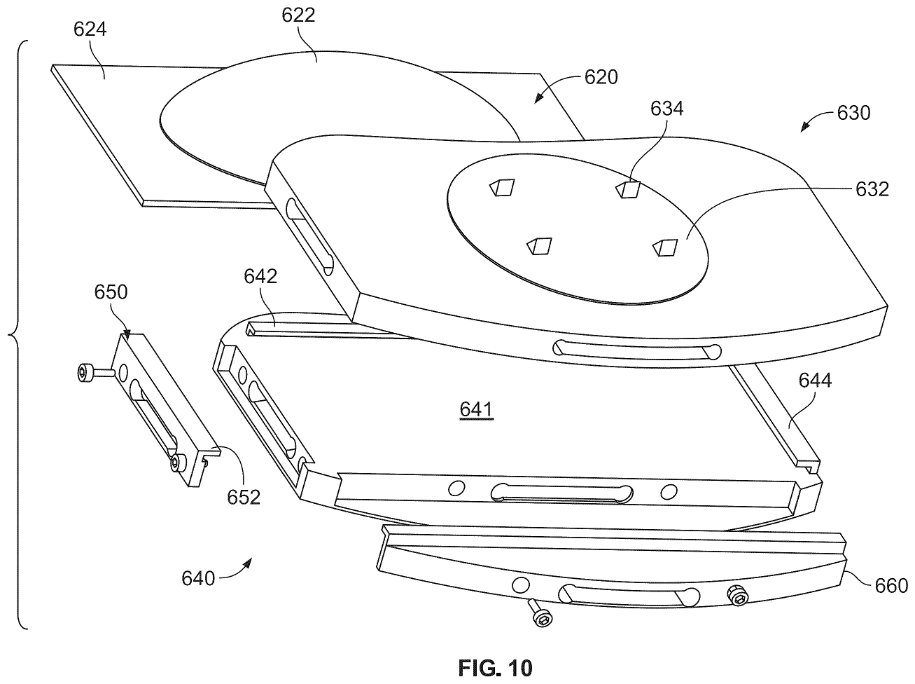

As shown in FIGS. 10-11A, core 620 has a semi-spherical tab 622 extending from a base 627. The tab 622 is received within a cavity (not shown) in the bottom surface of the upper plate 630. The tab 622 and cavity allow the upper plate 630 to articulate and flex relative to the core 620 and lower plate 640.

FIG. 11B is a front top perspective view of the lower plate 640 with the lateral connector 650 and anterior connector 660 removed. An exemplary groove, channel, or slot is shown on the left side of the plate (and generally depicted by reference numeral 644). The groove is defined in this embodiment by upper limb or rail 647 and the floor of the cavity 641. As described herein, the tongue portion 624 of the core is shaped to slidably engage the groove.

In contrast to the embodiments described in connection with FIGS. 1-8, core 620 has a substantially planar base 627 including an outer peripheral area 624 surrounding the tab 622. The outer peripheral area 624 surrounding the tab 622 is thin and, as described herein, can slidably engage parallel grooves (e.g., 644, 652) in the lower plate 640. In embodiments, the thickness (k) of the peripheral area 624 ranges from 1 to 6 mm, more preferably 2-4 mm. This tongue and groove arrangement between the core 620 and lower plate 640 allow the core to be conveniently installed and optionally revised using an anterior (and/or lateral) approach, discussed further herein.

The flat lower surface 427 of the core 420 adds stability to the disc assembly while still providing pivot motion between the upper plate relative to the core and lower plate. The combination of the flat base 427, cavity 641, and grooves 642, 644, 652, 662 prevent the core from slipping or reorientating after implantation in a patient. This is a meaningful advantage over artificial disc prothesis which lack redundant structures to maintain position of the core relative to the endplates.

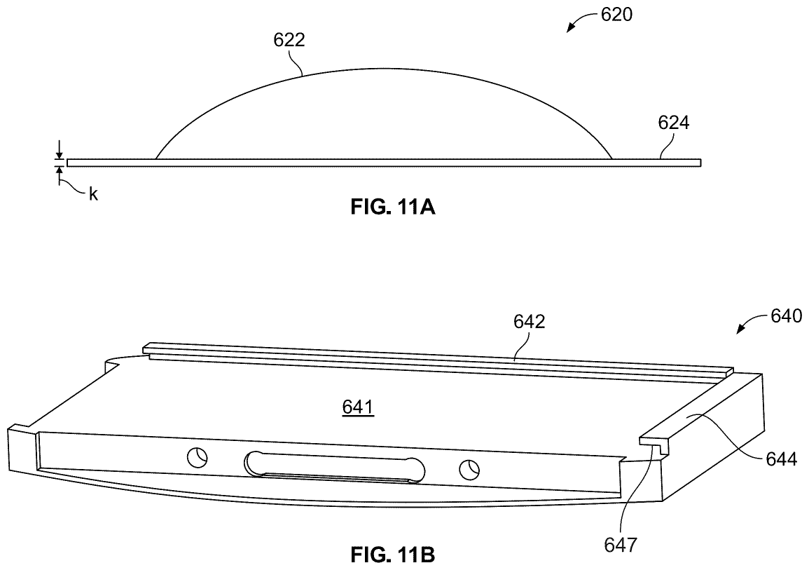

With reference to the embodiment shown in FIGS. 12-13, various exploded views of the core 620, lower plate 640, and anterior connector 660 are shown for illustrating an installation of the core in the lower plate. As shown, grooves 644, 652 of lower plate 640 are adapted to slidable receive lateral tongue portions 624 of the core 620 and guide the core into the cavity 641 defined between the grooves 644, 652, and posterior wall 642. After the core 620 is positioned in the cavity 641, anterior connector 660 is fastened to the lower end plate, locking the core in place.

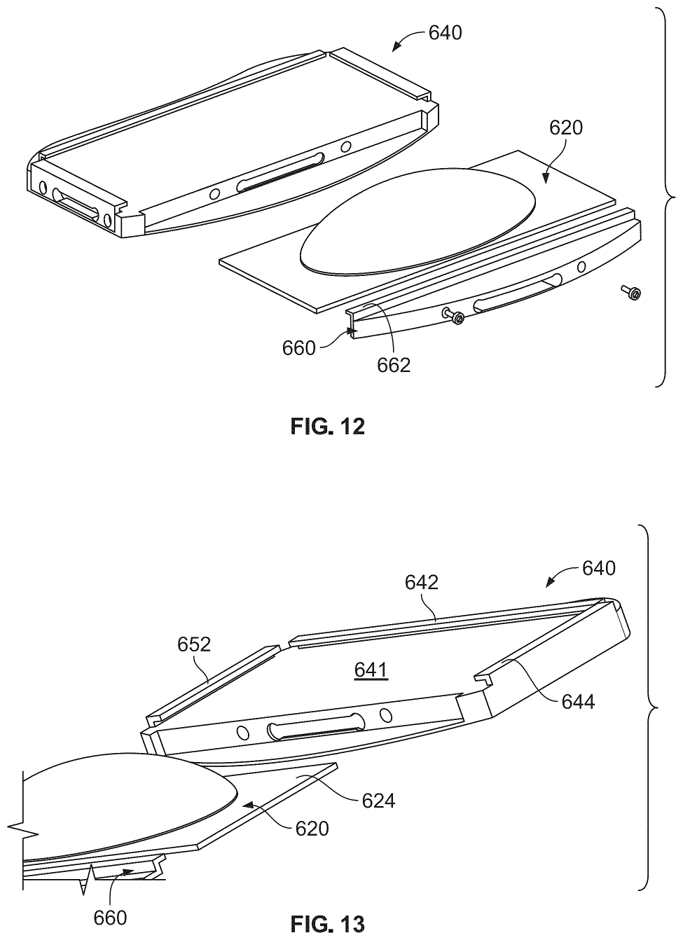

FIG. 14 is an enlarged partial perspective view of fastening the anterior connector 660 to the lower plate 640, and engaging the anterior connector 660 in the anterior recess defined by halfmoon/arcuate-shaped floor 646 and edge 648 of the lower plate. Examples of fasteners to lock the connector to the plate are screws 682, 684. Also shown is tool engagement feature in the form of an elongate slot 690/691 for receiving an end of a tool (not shown) to manipulate the connector.

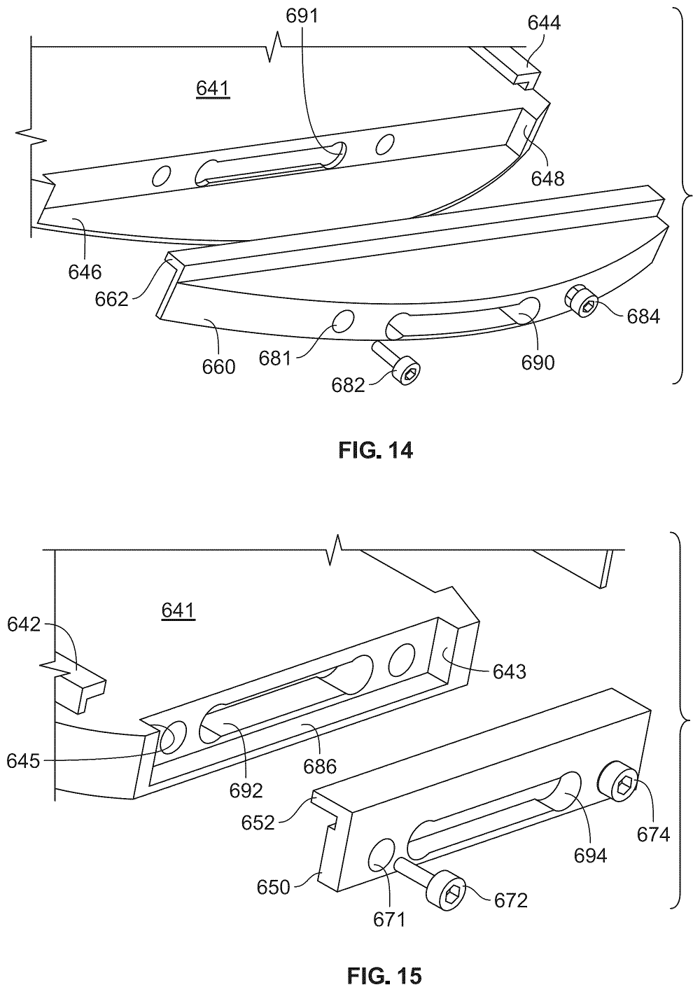

FIG. 15 illustrates an enlarged partial perspective view of fastening the left-side lateral connector 650 to the lower plate, and more specifically, engaging the lateral connector 650 in the left-side lateral recess defined by first edge 643, second edge 645, and floor 686 of the lower end plate. Examples of fasteners to lock the connector to the plate are screws 672, 674. Also shown is tool engagement feature in the form of an elongate slot 692, 694 for receiving an end of a tool (not shown) to manipulate the connector.

### (a) Initial Implantation—Anterior Approach

The initial artificial disc implantation may be performed using various approaches. For example, the initial artificial installation may use an anterior approach. In such a case, lower plate 640 is prepared by verifying the anterior connector 660 has been removed and the lateral connector 650 is securely fastened to the lower plate.

Next, the upper and lower end plates 630, 640 may be installed as described above in connection with FIG. 5.

Once the end plates are positioned between adjacent vertebrae in the spine, and spaced apart from one another, the core 620 is advanced into the lower plate by tracking parallel grooves 644, 652. After the core is located in the cavity 641 of the lower plate, the anterior connector 660 is fastened to the lower plate thereby securing the core in the lower plate.

Next, the upper plate 630 may be released and if needed, manipulated into engagement with the tab 622 of the core, resulting in the assembled disc 600 as shown in FIG. 9.

### (b) Initial Implantation—Lateral Approach

The initial artificial disc implantation may also be performed using a lateral approach. In such a case, lower plate 640 is prepared by verifying the lateral connector 650 has been removed and the anterior connector 660 is securely fastened to the lower plate.

Next, the upper and lower end plates 630, 640 may be installed as described above in connection with upper and lower plates of FIG. 5.

Once the end plates are positioned between adjacent vertebrae in the spine, and spaced apart from one another, the core 620 is advanced into the lower plate by tracking parallel grooves 642, 662. After the core is located in the cavity 641 of the lower plate, the lateral connector 650 is fastened to the lower plate thereby securing the core in the lower plate.

Next, the upper plate 630 may be released and if needed, manipulated into engagement with the tab 622 of the core, resulting in the assembled disc 600 as shown in FIG. 9.

### Revision Procedures

Similar to the revision procedure described above in connection with FIG. 6, the existing core may be replaced with a new core using various approaches.

### (a) Revision—Lateral Approach

In a lateral approach, after access to the existing disc is achieved, and the plates are spaced from one another, the lateral connector 650 is unfastened from the lower plate. In embodiments, it may be unfastened by unscrewing fasteners 672, 674.

The core 620 is then withdrawn from the cavity 641 along channels 642, 662. In embodiments, the core may be gripped by a tool such as a forceps, and guided out along the channels 642, 662.

A second core may then be installed as described above and the lateral connector refastened to the lower plate. The plates may then be released to snugly form around the core.

### (b) Revision—Anterior Approach

In an anterior approach, after access to the existing disc is achieved, and the plates are spaced from one another, the anterior connector 660 is unfastened from the lower plate. The core 620 is then withdrawn from the cavity 641 along channels 644,652. In embodiments, the core may be gripped by a tool such as a forceps, and guided out along the channels 644, 652.

A second core may then be installed as described above and the anterior connector refastened to the lower plate. The plates may then be released to snugly form around the core.

### Alternative Embodiments

Although a tongue-and-groove or ridge-and-slot arrangement is described above in connection with guiding the core in and out of the lower plate, the invention is not intended to be so limited. The core guide may vary widely and include one or more slots, tracks, guides, ridges, tongues, protrusions, and keys to slidably move and control one component relative to the other. Additionally, the cooperating features (tongue, ridge, groove, slot, channel, etc.) may be arranged on either the core or the plate. For example, the core may include a groove and the plate include the slidably cooperating tongue or ridge.

Throughout the foregoing description, and for the purposes of explanation, numerous specific details are set forth in order to provide a thorough understanding of the described techniques. It will be apparent, however, to one skilled in the art that these techniques can be practiced without some of these specific details. Although various embodiments that incorporate these teachings have been shown and described in detail, those skilled in the art could readily devise many other varied embodiments or mechanisms to incorporate these techniques. Also, embodiments can include various operations as set forth above, fewer operations, or more operations; or operations in another order than that specifically described above. Additionally, any of the components and steps described herein may be combined with one another in any logical manner except where such components or steps would be exclusive to one another. Accordingly, the scope and spirit of the invention should be judged in terms of the claims, which follow as well as the legal equivalents thereof.

1.-20. (canceled)

21. A method of revising an intervertebral disc prosthesis implanted via a first approach in a spine of a patient, the implanted intervertebral disc prosthesis comprising opposing plates and an existing core therebetween, the method comprising:
accessing the implanted intervertebral disc prosthesis in the spine of the patient;
spreading the plates from one another;
decoupling the existing core from the plates wherein the step of decoupling comprises slidably retracting the existing core along at least one groove in the lower plate;
removing the existing core from the patient while leaving the plates intact in the spine;
advancing a new core into the space between the plates wherein the step of advancing a new core into the space between the plates comprises slidably advancing the new core along the at least one groove in the lower plate; and
adjusting the new core and the plates such that the new core is aligned and pivotably engaged with the plates.

22.-45. (canceled)

46. The method of claim 21, wherein the step of decoupling comprises unfastening a connector from the lower plate prior to retracting the existing core.

47. The method of claim 46, wherein the existing core is laterally retracted.

48. The method of claim 47, wherein the connector is adapted to form a left or right side of the lower plate, and the at least one groove extends laterally (or from left to right).

49. (canceled)

50. (canceled)

51. The method of claim 21, further comprising fastening the connector to the lower plate after new core has been advanced into the lower plate, thereby securing the new core in the lower plate.

52. The method of claim 21, wherein the at least one groove comprises parallel grooves, forming a guide along which the new core is advanced or the existing core is retracted.

53. The method of claim 52, wherein the lower plate comprises a lateral connector and an anterior connector removably fastenable to the lower plate, and wherein the lateral connector forms a first groove when fastened to the lower plate, and the anterior connector forms a second groove when fastened to the lower plate, and wherein the first groove is perpendicular to the second groove.

54. The method of claim 45, wherein the revision is performed via a different approach than the first approach, and optionally the revision is performed from the left side of patient.

55.-60. (canceled)

61. An intervertebral disc prosthesis comprises:
an upper plate;
a lower plate;
a core adapted to be removably installed between the upper plate and the lower plate,
wherein the core comprises a substantially planar base, and a centrally-located superiorly-extending tab, and a tongue circumscribing the tab; and
wherein the lower plate comprises a left groove, a right groove, and a cavity between the left groove and the right groove, and wherein the left groove and right groove cooperate with a left portion and right portion of the tongue, respectively, of the base to slidably receive the base of the core in the cavity; and
an anterior connector removably engageable to an anterior side of the lower plate to secure the core in the cavity in the lower plate.

62. The intervertebral disc prosthesis of claim 61, wherein the anterior connector comprises a posterior facing rail, and forms an anteriorly-located groove for receiving an anterior portion of the tongue when the anterior connector is fastened to the lower plate.

63. The intervertebral disc prosthesis of claim 61, wherein the anterior connector further comprises at least one tool engagement feature to receive an end of a tool, and optionally, wherein the tool engagement feature is an elongate slot; and wherein the left-side connector further comprises at least one tool engagement feature to receive an end of a tool, and optionally, wherein the tool engagement feature is an elongate slot.

64. The intervertebral disc prosthesis of claim 61, wherein the left groove is formed by a rail of a left-side connector removably engageable to the left side of the lower plate.

65. (canceled)

66. The intervertebral disc prosthesis of claim 61, wherein the right groove is formed by a rail of a right-side connector removably engageable to the right side of the lower plate.

67. The intervertebral disc prosthesis of claim 61, wherein the lower plate further comprises a posterior groove adapted to slidably engage a posterior portion of the tongue.

68. The intervertebral disc prosthesis of claim 61, wherein the tab of the core has a semispherical shape and an inner surface of the upper plate comprises a concave recess to pivotably cooperate with the tab when the disc is assembled in the body.

69. (canceled)

70. The intervertebral disc prosthesis of claim 61, wherein the upper surface of the upper plate comprises a bow, and optionally at least one fin.

71. The intervertebral disc prosthesis of claim 61, wherein the upper plate and lower plate each further comprise at least one tool slot or hole arranged on an anterior surface, a lateral surface, and optionally both the anterior and lateral surface.

72. (canceled)

73. The intervertebral disc prosthesis of claim 61, wherein the upper plate comprises at least one tool engagement feature on an anterior or lateral side, and wherein the at least one tool engagement feature is operable to receive an end of a tool, and optionally, wherein the tool engagement feature is an elongate slot.

74. The intervertebral disc prosthesis of claim 61, wherein the ratio of the thickness of the tongue to maximum height of the core ranges from 2-20%, optionally, 5-15%, and in some embodiments, 5-10%.

75.-94. (canceled)

95. A method of revising a core of an artificial intervertebral disc prothesis comprising sliding out an existing core along a first guide in a lower plate; and
tracking a new core along the first guide into the lower plate.

96. The method of claim 95, further comprising, after the advancing step, installing a connector to the lower plate to secure the core in the lower plate.

97. The method of claim 96, wherein the connector comprises a slot operable as a second guide for the core in a direction different than the first guide.

98. (canceled)
